(19) United States
(12) Patent Application Publication
(10) Pub. No.: US 2016/0198967 A1
(43) Pub. Date: Jul. 14, 2016

(54) METHOD AND SYSTEM FOR DETECTING SIGNALS OF PULSE DIAGNOSIS, AND DETECTING DEVICE OF PULSE DIAGNOSIS

(71) Applicant: TAIDOC TECHNOLOGY CORPORATION, New Taipei City (TW)

(72) Inventor: Wei-Chiang Hsu, New Taipei City (TW)

(21) Appl. No.: 14/991,407

(22) Filed: Jan. 8, 2016

(30) Foreign Application Priority Data
Jan. 9, 2015 (TW) 104100614

(51) Int. Cl. A61B 5/024; A61B 5/00

(52) U.S. Cl. CPC A61B 5/02444; A61B 5/0022; A61B 5/6825; A61B 5/743

(57) ABSTRACT

The present invention is related to a method and a system for detecting signals of pulse diagnosis, and a detecting device of pulse diagnosis. The method comprises following steps. Apply a pressure on an artery of an object, and then detect a pulsation generated from a feedback of the artery at each different pressure value. Compare the similarity of a preset period and a preset vibration amplitude of the artery with a default sphygmogram model at each different pressure value to determine a pressure value of pulse diagnosis by a graphical analog method, and obtain a corresponding sphygmogram according to the pressure value of pulse diagnosis. It will provide standardization and increase the data applicability for personal pulse wave analysis by comparing personal different sphygmogram to determine different pressure value of pulse diagnosis.

### BACKGROUND OF THE INVENTION

1. Field of the Invention

The invention relates to a method and a system for detecting signals of pulse diagnosis, and a detecting device of pulse diagnosis. It particularly relates to a method and a system for detecting signals of pulse diagnosis, and a detecting device of pulse diagnosis to determine the detecting pressure value of the pulse diagnosis by comparing the sphygmomanometer by graphical analog method.

2. Description of the Related Art

Traditional Chinese Medicine (TCM) uses four diagnostic methods which is observing, listening, asking, and feeling and differentiating the symptoms to diagnose illness, wherein feeling pulse is one of the important clinical diagnosis means by placing the fingers to the patients' Radial artery of the wrist by Chinese medical doctors. FIG. 1 shows the positions of cun, guan, and chi on human wrist according to the present invention in a schematic view. Please refer to FIG. 1. In terms of TCM point of view, press cun, guan and chi by using three fingers respectively and feel the vascular elasticity and pressure state to diagnose different pulse conditions by applying different pressures, and the depth of applying the pressure from small to large sequentially called floating pulse taking, medium pulse taking, deep pulse taking, and collectively called three depths taking.

The pulse diagnosis is based on the tactile acuity of Chinese medical doctor's fingers, the accumulated experiences of pulse diagnosis, and the theories record in ancient books. On the one hand, it cannot quantify the depth of the applying pressure, and easy to affect diagnostic results by personal subjective consciousness. On the other hand, it is difficult to do statistical analysis due to the lack of objective data. Therefore, the scientization of TCM is a long time issue hoped to be resolved.

In the perspective of modern medicine, the pulse condition is mainly constituted by the heartbeat. Therefore, the detecting device by using the theory of detecting blood pressure to collect the pulse condition of wrist becomes one of the modern trends of pulse diagnosis device. However, if it accesses data of blood pressure while detecting only in western medicine perspective, then there is no correlation with the pulse wave in TCM. Moreover, the conventional setup pressure value of three depths taking in pulse diagnosis is a default value or a blood pressure-related parameter; it does not meet the features of TCM diagnosis perspective of pulse condition, and also cannot reflect the feature of personal pulse condition if it simply standardized collecting data of pulse.

Thus, for how to provide the standardized pulse wave collection by using modern detecting device, and determining the applying pressure value of the subject's three depths taking based on the personal pulse difference to facilitate distinguishing personal pulse condition, and providing TCM to do the various kinds of analysis are what the manufacturers need to be further improved.

### SUMMARY OF THE INVENTION

In order to improve the conventional deficiencies above, the present invention provides a method to screen personal pulse data and determine an applying pressure value of pulse diagnosis by graphical analog method, then to compare a default value or a blood pressure-related parameter to be more in line with a sphygmogram demand in TCM.

The present invention provides a method for detecting signals of pulse diagnosis to determine a pressure value of pulse diagnosis by using a graphical analog method based on a data of personal pulse, and then to collect the sphygmogram from the pressure value of pulse diagnosis to distinguish different features of pulse individually to apply designated pressure, and to provide a personal pressure value of pulse diagnosis, then to obtain a data of pulse wave to do the related analysis.

The present invention provides a detecting device of pulse diagnosis disposed at the wrist or the arm of the user to let the cuff inflation or deflation, fix the pulse collecting position of the detecting device, determine the range of the pressure value of pulse collection based on the personal pressure value of pulse diagnosis, exclude the human judgment factor causing the position difference of pulse taking, and increase the reliability of the digitization of personal pulse diagnosis by using standardized methods.

The present invention provides a system for detecting signals of pulse diagnosis actuated the transmission function of a sphygmogram of a detecting device of pulse diagnosis by using an external device which is beneficial to have effective analysis and instantaneous diagnosis of personal sphygmogram for telemedicine doctors.

In order to achieve the purpose above, the present invention provides a method for detecting signals of pulse diagnosis, comprising:

applying a pressure on an artery of an object;

detecting a pulsation generated from a feedback of the artery at each different pressure value;

comparing the similarity of a preset period and a preset vibration amplitude of the pulsation at each different pressure value with a default sphygmogram model to determine a pressure value of pulse diagnosis by a graphical analog method; and

obtaining a corresponding sphygmogram according to the pressure value of pulse diagnosis.

In an embodiment in accordance with the present invention, the pressure value of pulse diagnosis can comprise a pressure value of deep pulse taking, a pressure value of medium pulse taking, and a pressure value of floating pulse taking, and the at least two pressure values are determined by changing the other pressure value with a default range.

In an embodiment in accordance with the present invention, the pressure value of deep pulse taking and the pressure value of floating pulse taking are determined by the pressure value of medium pulse taking adding or subtracting the default range.

In an embodiment in accordance with the present invention, the default range can be 15 mmHg.

In an embodiment in accordance with the present invention, obtaining a corresponding sphygmogram according to the pressure value of pulse diagnosis is obtained the corresponding sphygmogram from large to small sequentially of the pressure value of pulse diagnosis.

In an embodiment in accordance with the present invention, obtaining a corresponding sphygmogram is obtaining a corresponding sphygmogram collected from maintaining the pressure value of pulse diagnosis during a default time.

In an embodiment in accordance with the present invention, the preset period of the pulsation comprises rapid ventricular ejection, left ventricular contraction and left ventricular relaxation. In an embodiment in accordance with the present invention, the preset vibration amplitude of the pulsation comprises percussion wave, height of the dicrotic notch and dicrotic wave.

In an embodiment in accordance with the present invention, the method for detecting signals of pulse diagnosis can further comprise transmitting the sphygmogram to the internet or a data receiver.

From another point of view, the present invention provides a detecting device of pulse diagnosis detected a signal of pulse diagnosis by a pulsation generated from an artery of an object, comprising:

a cuff disposed on the object to pressurize while inflation and decompress while deflation;

a detector for detecting the pulsation generated from a feedback of the artery pressurized by the cuff, and obtaining a sphygmogram at each different pressure value;

a microprocessor coupled with the detector for comparing the similarity of a preset period and a preset vibration amplitude of the pulsation at each different pressure value with a default sphygmogram model to determine a pressure value of pulse diagnosis by a graphical analog method, and then to obtain a corresponding sphygmogram according to the pressure value of pulse diagnosis.

In an embodiment in accordance with the present invention, the detecting device of pulse diagnosis can be a wrist type detecting device.

In an embodiment in accordance with the present invention, the detecting device of pulse diagnosis can further comprise:

an air pump connected to the cuff to pressurize the cuff while inflation;

a controller coupled with the cuff to control the pressure value inside the cuff; and

a pressure sensor coupled with the detector and the microprocessor to convert the pulsation to an electrical signal and provide the preset period and the preset vibration amplitude information of the pulsation.

In an embodiment in accordance with the present invention, the detecting device of pulse diagnosis can further comprise a transmitter coupled with the microprocessor to transmit the corresponding sphygmogram to the internet or a data receiver.

In an embodiment in accordance with the present invention, the detecting device of pulse diagnosis can further comprise a display coupled with the microprocessor to display the sphygmogram and/or the related status information of the pulse diagnosis detecting device.

From another point of view, the present invention provides a system for detecting signals of pulse diagnosis, comprising:

a detecting device of pulse diagnosis as above; and

an external device comprising an actuator and a data receiver, wherein the actuator is used for controlling turning on or off the transmission function of the sphygmogram of the detecting device of pulse diagnosis.

Based on the described above, a method and a system for detecting signals of pulse diagnosis and a detecting device of pulse diagnosis of the present invention obtained personal data of pulse wave under different pressures by inflation and deflation the cuff, determined a specific pressure of pulse diagnosis by graphical analog method to compare a single preset period and vibration amplitude of sphygmogram, further collecting the sphygmogram data under the pressure value, and provided the data which TCM needed by the standardized method of pulse collecting condition.

In order to make the features and the advantages more realizable in the present invention, the following description and accompanying drawings are some examples in accordance with the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the positions of cun, guan, and chi on human wrist according to the present invention in a schematic view;

FIG. 2 shows a preferred embodiment of a system for detecting signals of pulse diagnosis of the present invention in a functional block diagram;

FIG. 3 shows a preferred embodiment of a detecting device of pulse diagnosis of the present invention in a detecting output sphygmogram;

FIG. 4 shows a preferred embodiment of a detecting device of pulse diagnosis of the present invention in a sphygmogram model;

FIG. 5 shows a preferred embodiment of a device for detecting signals of pulse diagnosis of the present invention in a flowchart;

FIG. 6 shows another preferred embodiment of a device for detecting signals of pulse diagnosis of the present invention in a flowchart;

FIG. 7 shows another preferred embodiment of a detecting device of pulse diagnosis of the present invention in a detecting output sphygmogram.

### DESCRIPTION OF THE PREFERRED EMBODIMENTS

A conventional pulse diagnosis of TCM is easy to be affected by the tactile acuity of Chinese medical doctors' fingers and the different experiences of pulse diagnosis. Especially, it cannot provide an unified quantitative standard because the varies of the position and the pressure of pulse taking. Even though there is a wrist type detecting device of pulse diagnosis by using the theory of detecting blood pressure in modern times, but the applying pressure of three depths taking uses a default value or a blood pressure-related parameter to setup, and it collects sphygmogram information simply in western medicine perspective, therefore it cannot show the applying pressure change of digitization information according to the personal pulse condition.

On the contrary, an embodiment of the present invention collects the data of personal pulse wave under different pressures, and determined the pressure value of pulse diagnosis by graphical analog method to compare a single preset period and vibration amplitude of sphygmogram, and then to adjust the value of the applied three depths taking pressure according to the features of personal pulse, and last to collect the objective and digitization sphygmogram in the perspective of TCM pulse in accordance with the present invention. The following detailed description and accompanying drawings are some examples in accordance with the present invention. The same symbol herein in the drawings indicates the same or similar structure.

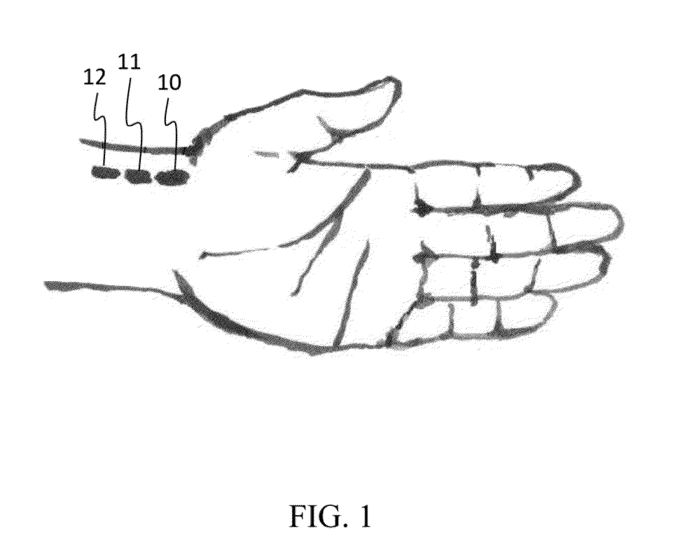

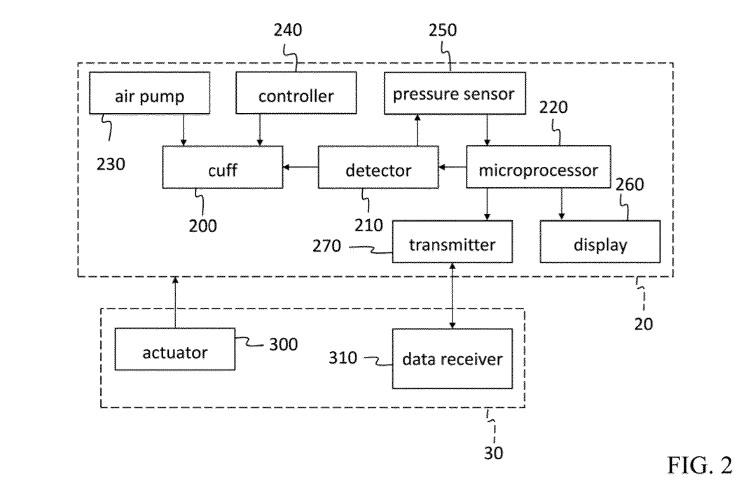

FIG. 2 shows a preferred embodiment of a system for detecting signals of pulse diagnosis of the present invention in a functional block diagram. Please refer to FIGS. 1 and 2 in combination. In the present embodiment, a system for detecting signals of pulse diagnosis comprises a detecting device of pulse diagnosis (20) and an external device (30), preferably, the detecting device of pulse diagnosis can be a wrist or an arm type sphygmomanometer, but the present invention shall not be limited in this. Those skilled in the art can change the different positions of human body where the detecting device of pulse diagnosis disposed on as needed, and the following will use the wrist type sphygmomanometer for the description. The detecting device of pulse diagnosis (20) comprises a cuff (200), a detector (210), and a microprocessor (220), preferably, the detecting device of pulse diagnosis (20) can be further included but not limited to an air pump (230), a controller (240), a pressure sensor (250), a display (260), and a transmitter (270). The cuff (200) disposed on an object to pressurize while inflation and decompress while deflation, preferably, the cuff (200) can be disposed on the wrist or the arm of human body, more preferably, the cuff (200) can be disposed at the positions of can (10), guan (11), and chi (12) on the wrist of human body. More specifically, apply a pressure on an artery of an object or a wrist of human body to block the blood flow of the artery while the cuff (22) is pressurized. On the contrary, it will continue reducing oppression of the artery and generating a gradually increasing blood flow in the artery while the cuff (200) is decompression.

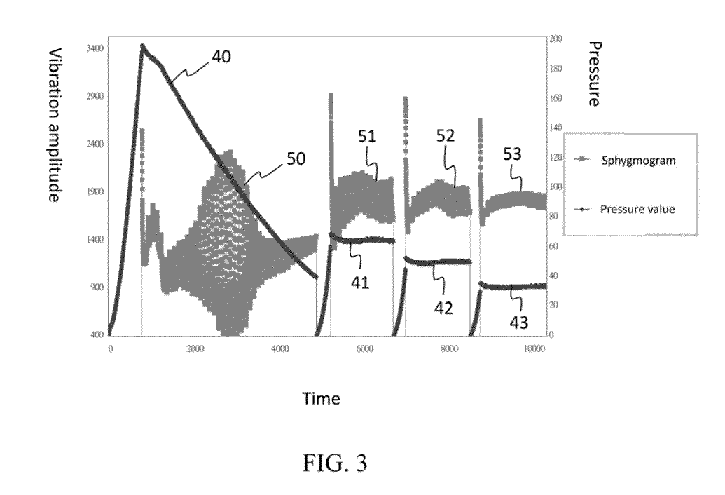

The detector (210) for detecting a pulsation generated from a feedback of the artery which is applied pressure by the cuff (200), and obtained a pulse wave at each different pressure value, preferably, the type of the detector (210) can be included but not limited to resistive pressure sensing unit or capacitive pressure sensing unit. FIG. 3 shows a preferred embodiment of a device for detecting signals of pulse diagnosis of the present invention in a detecting output sphygmogram. Please further refer to FIG. 3, the pressure value of cuff (40) is pressurized while inflation, and deflation to decompress when reach to a constant value. With the change of different downward pressure values will form a feedback and obtain a sphygmogram of pulsation (50) from each arterial beat while the cuff (200) is decompression.

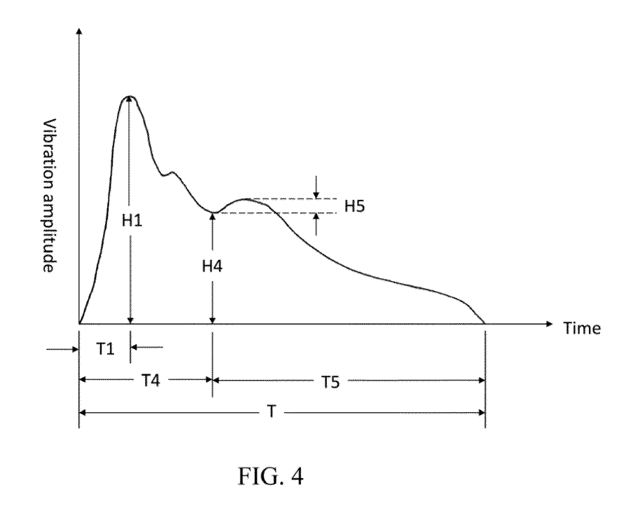

The microprocessor (220) is coupled with the detector (210) for comparing the similarity of a preset period and a preset vibration amplitude of the artery with a default sphygmogram model at each different pressure value, so as to determine a pressure value of pulse diagnosis by a graphical analog method, and then obtain a corresponding sphygmogram according to the pressure value of pulse diagnosis. FIG. 4 shows a preferred embodiment of a device for detecting signals of pulse diagnosis of the present invention in a sphygmogram model. Please further refer to FIG. 4. In the present embodiment uses a wrist type sphygmogram as an example, but the present invention shall not be limited in this. The sphygmogram is a sphygmogram of a single pulse which is defined by different parameters of the period and vibration amplitude, the parameter of the period comprises a period of the pulsation (T), rapid ventricular ejection (T1), left ventricular contraction (T4), and left ventricular relaxation (T5), wherein the rapid ventricular ejection (T1) is the moment that the blood reaches the maximum pressure within the heart in left ventricular contraction (T4), and force heart valve to open and let the blood eject then flow into the aorta.

It is worth mentioning that, a pulse formation is mainly relying on the systole and the diastole of the heart, and the distensibility and elasticity of arterial wall. The sphygmogram is constituted by ascending branch and descending branch, the ascending branch is mainly manifested at passive distension of arterial wall in rapid ventricular ejection (T1), and the descending branch is mainly manifested at the distension of aorta which is started to retract after rapid ventricular ejection (T1). With the decreasing of blood ejection, the pressure decline within ventricular and result in the reflux of aortic blood and further caused the aortic valve closed, then form the position of height of the dicrotic notch (H4) on sphygmogram. It blocks the reflux of the blood due to the aortic valve closed, and let the blood continue flowing to the vascular end, then forming a reflexed up of the dicrotic wave (H5). It can compare the difference of the wave type and the appearance time of peaks and troughs in the sphygmogram to reflect the personal pulse condition and the features of constitution, so the tracing record of the sphygmogram is beneficial to combine the clinical experiences of feeling pulse of TCM, then make the digitization statistical analysis objectively. The method for detecting signals of pulse diagnosis will be described in detail later.

The air pump (230) is connected to the cuff (200) and pressurized to the cuff (200) while inflation. The controller (240) is coupled with the cuff to control the pressure value inside the cuff. More specifically, it can control the pressure releasing speed and level of the cuff (200) by the controller (240) when the cuff (200) reached to a constant value, preferably, the controller (240) can be a control valve. The pressure sensor (250) is coupled with the detector (210) and the microprocessor (220) to convert the pulsation to an electrical signal and provide the preset period and the preset vibration amplitude information of the pulsation, preferably, the pressure sensor (250) can be an analog/digital converter unit.

The display (260) is coupled with the microprocessor (220) to display the sphygmogram and/or the related status information of the pulse diagnosis detecting device. For example, the related status information of the pulse diagnosis detecting device can be included but not limited to the state-of-charge (SOC), the pressure value of pulse diagnosis, the pressure value of the cuff, the setting date or time, or the error message etc. Besides, the display (260) can use voice, image, number, symbol or light etc., the different types of interface to express the information of above, but the present invention shall not be limited in this. For example, the display (260) is a liquid crystal screen for displaying the sphygmogram, and using the battery symbol to represent the SOC. Another example, the display (260) is a speech unit which speaks out the detecting step of the pulse diagnosis or the pressure value of the pulse diagnosis.

The transmitter (270) is coupled with the microprocessor (220) to transmit the corresponding sphygmogram to the internet or a data receiver (310), preferably, the type of the transmitter (270) can be wired or wireless transmission. For example, the wired transmission can be RS232 or USB, and the wireless transmission can be Bluetooth, infrared transmission, Wi-Fi, local area network (LAN), Internet etc. and any other wireless transmission techniques understanding by those skilled in the art.

The external device (30) comprises an actuator (300) and a data receiver (310), the actuator (300) is for controlling the turning on or off of the transmission function of a sphygmogram of the pulse diagnosis detecting device (20), preferably, the external device (30) is desktop computer, mobile phone, palmtop computer or laptop, but the present invention shall not be limited in this. More specifically, the detecting device has a signal detection of pulse diagnosis while the subject is wearing the detecting device of pulse diagnosis (20). After determined the pressure value of the pulse diagnosis, the actuator (300) will turn on the transmission function of a sphygmogram of the pulse diagnosis detecting device and received a corresponding sphygmogram detecting under the pressure value of the pulse diagnosis by the data receiver (310), and let the telemedicine doctors can analyze the data instantly, but the present invention shall not be limited in this. Those skilled in the art can change the timing or the period of transmission function with regards to detecting device of pulse diagnosis (20), and data receiver (310) receives the transmission content from the detecting device of pulse diagnosis (20) as needed.

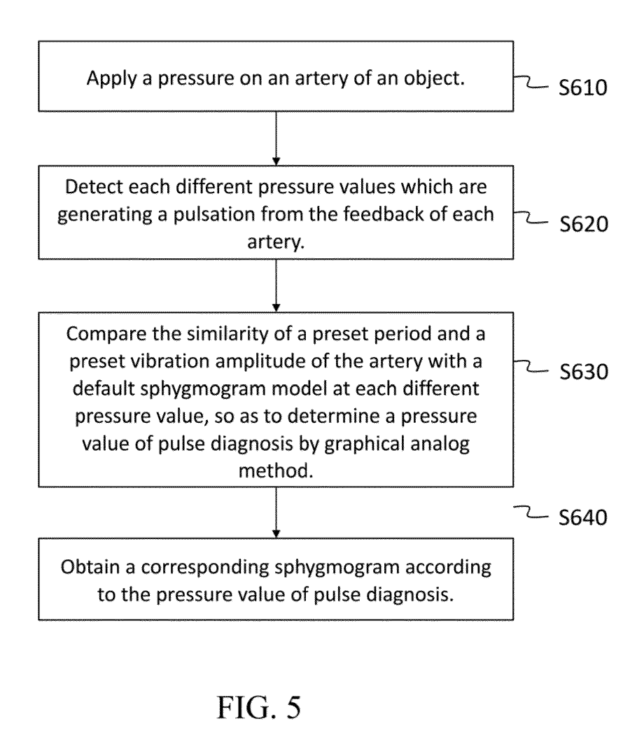

FIG. 5 shows a preferred embodiment of a device for detecting signals of pulse diagnosis of the present invention in a flowchart. Please refer to FIGS. 2 to 5 in combination. In the present embodiment, firstly, apply a pressure on an artery of an object in the step S610, more preferably, the source of the pressure will use the cuff (200) of the detecting device of the pulse diagnosis (20) as an example for the description, but the present invention shall not be limited in this. More preferably, the cuff (200) is applying a pressure on an artery of an object by the air pump (230) to block the blood flow of the artery, and while the cuff (200) is pressurized and reach to a constant value, the controller (240) will let the cuff (200) decompress gradually while deflation to reduce the oppression on the artery, and then generating a gradually increasing blood flow.

Detect each pulsation from each different pressure values respectively which is generating from the feedback of each artery in step S620. More specifically, through applying different pressure values to the cuff (40) will let the artery generate a feedback and form a continuous sphygmogram of pulsation (50) in the decompression process, more preferably, the pressure sensor (250) will convert the pulsation into an electrical signal and provide the preset period and the preset vibration amplitude information of the pulsation to the microprocessor (220). More preferably, the parameter of the period can be included but not limited to the pulsation (T), rapid ventricular ejection (T1), left ventricular contraction (T4), and left ventricular relaxation (T5), and the parameter of the vibration amplitude can be included but not limited to percussion wave (H1), height of the dicrotic notch (H4), and dicrotic wave (H5).

Compare a similarity of a preset period and a preset vibration amplitude of the pulsation with a default sphygmogram model at each different pressure value, so as to determine a pressure value of pulse diagnosis by a graphical analog method in step S630. More specifically, the step is comparing the sphygmogram of each single period from the obtained sphygmogram of pulsation (50) under different pressure values of cuff (40) with the default sphygmogram model (as shown in FIG. 4) by using the graphical analog method, and more preferably, the step is comparing the type of the pulse wave by amplitude node. For example, the vibration amplitude starting point of the pulsation (T) is Point 1, the vibration amplitude of the percussion wave (H1) is Point 2, the vibration amplitude of the height of the dicrotic notch (H4) is Point 3, the vibration amplitude of the dicrotic wave (H5) is Point 4, and the vibration amplitude ending point of the pulsation (T) is Point 5, and then to compare the similarity between the feature node of Point 1 to Point 5 and each single period of pulse wave, but the present invention shall not be limited in this. Those skilled in the art can change the feature of identifying the type of the pulse wave as needed. For example, the similarity of line segment, curve, angle and slope in the coordinate axis, and choose the most pressure value of the pressure range which meets the default sphygmogram model and set as the pressure value of the pulse diagnosis of the subject. In other words, the graphical analog method is to compare the similarity between each detecting output sphygmogram and the sphygmogram model, preferably, the pressure value of pulse diagnosis can be included but not limited in a pressure value of floating pulse taking, a pressure value of medium pulse taking, and a pressure value of deep pulse taking.

Obtain a corresponding sphygmogram according to the pressure value of pulse diagnosis in step S640. For example, the pressure value of pulse diagnosis is 50 mmHg, the cuff (200) of the detecting device of pulse diagnosis (20) is re-pressurized to 50 mmHg and maintained the pressure value to collect corresponding sphygmogram during a default time, preferably, the default time is ten seconds. Moreover, preferably, the sphygmogram collected from the corresponding pressure value of pulse diagnosis can transmit to the internet or a data receiver, and provide the doctor to do the instantly analysis and diagnosis.

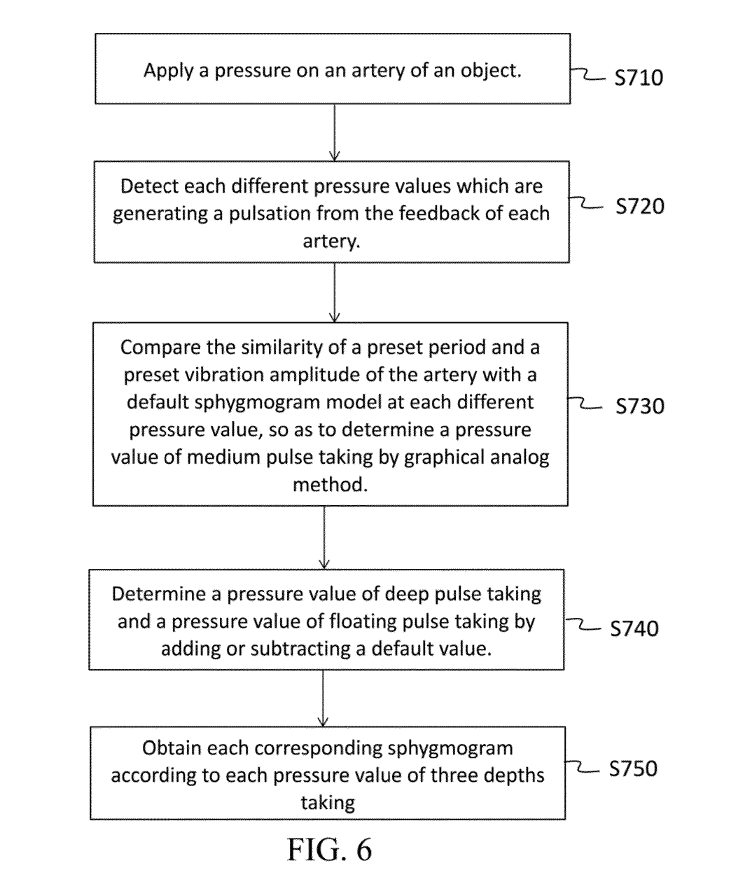

FIG. 6 shows another preferred embodiment of a device for detecting signals of pulse diagnosis of the present invention in a flowchart. Please refer to FIGS. 2 to 4 and 6 in combination. In the present embodiment, firstly, applying a pressure on an artery of an object in step S710, detecting a pulsation generated from a feedback of the artery at each different pressure value in step S720, and then to compare the similarity of a preset period and a preset vibration amplitude of the artery with a default sphygmogram model at each different pressure value, so as to determine a pressure value of medium pulse taking by graphical analog method in step S730 which is only an alternative embodiment of the present invention. In accordance with the other embodiments, the pressure value of medium pulse taking can also be a pressure value of deep pulse taking or a pressure value of floating pulse taking.

Based on the pressure value of the medium pulse to determine a pressure value of deep pulse taking and a pressure value of floating pulse taking by adding or subtracting a default value in step S740, preferably, the default pressure value is 15 mmHg. For example, the pressure value of the medium pulse taking is 50 mmHg and the default value is 15 mmHg so the pressure value of deep pulse taking and the pressure value of floating pulse taking will be 65 mmHg and 35 mmHg respectively, but the present invention shall not be limited in this, those skilled in the art can change the default pressure range as needed.

Obtain each corresponding sphygmogram according to each pressure value of three depths taking in step S750, preferably, the corresponding sphygmogram is sequentially obtained from large to small of the pressure value of pulse diagnosis. For example, pressure value of deep pulse taking (41), pressure value of medium pulse taking (42) and the pressure value of floating pulse taking (43) is 65 mmHg, 50 mmHg and 35 mmHg respectively. The cuff (200) is re-pressurized to 65 mmHg, and maintained the pressure value of deep pulse taking (41) to collect corresponding sphygmogram of deep pulse taking (51) during a default time. When it reaches to the default time, the controller (240) will let the cuff (200) decompress while deflation and re-pressurized to 50 mmHg, and then to maintain the pressure value of medium pulse taking (42) to collect corresponding sphygmogram of medium pulse taking (52) during a default time. When it reaches to the default time, the controller (240) will let the cuff (200) decompress while deflation and re-pressurized to 35 mmHg, and then to maintain the pressure value of floating pulse taking (43) to collect corresponding sphygmogram of floating pulse taking (53) during a default time.

Although the possible kinds of the method and the system for detecting signals of pulse diagnosis, and the detecting device of pulse diagnosis in accordance with the present invention has been described in the embodiments above, those skilled in the art shall recognized that the method and the system for detecting signals of pulse diagnosis, and the detecting device of pulse diagnosis can be designed differently. Therefore, the spirit of the present invention shall not be limited to these possible kinds of method and system for detecting signals of pulse diagnosis, and the detecting device of pulse diagnosis in accordance with the present invention. In other words, comparing the similarity of the personal sphygmogram under different pressures and default sphygmogram models by graphical analog method to determine the applying pressure value of pulse diagnosis and obtained the corresponding sphygmogram which is the key spirit and scope of the present invention. The followings are some other embodiments in accordance with the present invention for those skilled in the art to know more about the spirit of the present invention.

The embodiments in accordance with FIG. 2 described as above, the detecting device of pulse diagnosis (20) comprises a display (260), and the display (260) is coupled with the microprocessor (220) which is only an alternative embodiment of the present invention. In accordance with the other embodiments, the display (260) also can dispose at the external device (30), those skilled in the art can change the disposed position of the display (260) as needed.

Following the description above, the detecting device of pulse diagnosis (20) comprises a transmitter (270) for transmitting the corresponding sphygmogram to the internet or a data receiver (310) which is only an alternative embodiment of the present invention. In accordance with the other embodiments, the transmitter (270) can also transmit the related status information of the detecting device of pulse diagnosis (20). For example, the related status information of the pulse diagnosis detecting device can be the state-of-charge (SOC), the pressure value of pulse diagnosis, the pressure value of the cuff, the setting date or time, or the error message. Those skilled in the art can change the transmission content of the transmitter (270) as needed.

The embodiments in accordance with FIG. 2 described as above, the system for detecting signals of pulse diagnosis comprises a detecting device of pulse diagnosis (20) and an external device (30), the external device (30) comprises an actuator (300) to control turning on or off of the transmission function of a sphygmogram of the pulse diagnosis detecting device which is only an alternative embodiment of the present invention. In accordance with the other embodiments, the actuators can also use for actuating the detecting device of pulse diagnosis itself or controlling the pressure value of the cuff. Those skilled in the art can change the corresponding type of the detecting device of pulse diagnosis controlling by the actuator as needed.

The embodiments in accordance with FIGS. 4 and 5 described as above, compare the similarity of a preset period and a preset vibration amplitude of the artery with a default sphygmogram model at each different pressure value, so as to determine a pressure value of pulse diagnosis by graphical analog method in step S630, and the default sphygmogram model uses the FIG. 4 as an example for the description which is only an alternative embodiment of the present invention. Those skilled in the art can change the graphic features of the default sphygmogram model as needed. For example, the point, line segment, curve, angle and slope or other parameters can be used.

The embodiments in accordance with FIG. 6 described as above, based on the pressure value of the medium pulse to determine a pressure value of deep pulse taking and a pressure value of floating pulse taking by adding or subtracting a default value in step S740 which is only an alternative embodiment of the present invention. In accordance with the other embodiments, the pressure value of three depths taking can determine other two pressure values by changing a default range according to one of the pressure value. For example, the pressure value of deep pulse taking is 70 mmHg determined by the graphical analog method and the default range is 20 mmHg. Base on the pressure value of deep pulse taking, lower the default range or the multiples of the default range to determine the pressure value of medium pulse taking and the pressure value of floating pulse taking, so the pressure value of medium and floating pulse taking are 50 mmHg and 30 mmHg respectively. Similarly, it can also determine the pressure value of floating pulse taking by the graphical analog method. Take 40 mmHg as an example of the floating pulse taking to determine the pressure value of medium and deep pulse taking, and the pressure value of medium and deep pulse taking are 60 mmHg and 80 mmHg when the default range is 20 mmHg.

Following the description above, obtain each corresponding sphygmogram according to each pressure value of three depths taking in step S750, preferably, the corresponding sphygmogram is sequentially obtained from large to small of the pressure value of pulse diagnosis. The controller (240) is according to the pressure value of deep pulse taking (41), pressure value of medium pulse taking (42) and the pressure value of floating pulse taking (43) to let the cuff re-pressurized and decompress at different times which is only an alternative embodiment of the present invention. In accordance with the other embodiments, the controller can use the staged decompression way to pressurize to the pressure value of the deep pulse taking, and decompress gradually to the pressure value of the medium pulse taking after collecting the sphygmogram of deep pulse taking, and decompress gradually to the pressure value of the floating pulse taking after collecting the sphygmogram of medium pulse taking. Those skilled in the art can change the circulation way of pressurizing and decompressing of the cuff as needed.

The embodiments in accordance with FIGS. 5 and 6 described as above, apply a pressure on an artery of an object to block the blood flow of the artery, then continue reducing oppression of the artery and generate a gradually increasing blood flow in the artery while the cuff is releasing pressure in step S610 and S710. Detect each different pressure values which are generating a pulsation from the feedback of each artery in step S620 and S720. In other words, the process described as above is a deflation way of detecting which is only an alternative embodiment of the present invention. More specifically, applying the pressure on the artery can change the opening and closing state of the path of blood flow in the artery to obtain the feedback of the artery at different pressure value. For example, in the deflation way of detecting process, the different pressure value is obtained from the artery at the diameter of the arterial vessel from full closure state, gradually to half opened and at last to full open state (as shown in FIG. 3). On the contrary, in the inflation way of detecting process, the different pressure value is obtained from the artery at the diameter of the arterial vessel from full open state, gradually to half opened and at last to full closure state. It is worth noting that the inflation way does not need to pressurize the diameter of the arterial vessel to full closure and just need to meet the pressure value of the most similar default sphygmogram as the goal to stop pressurizing, hereafter to decompress while deflation. Therefore, those skilled in the art can choose to detect the pulsation at each different pressure value during the process of pressurized or decompression as needed.

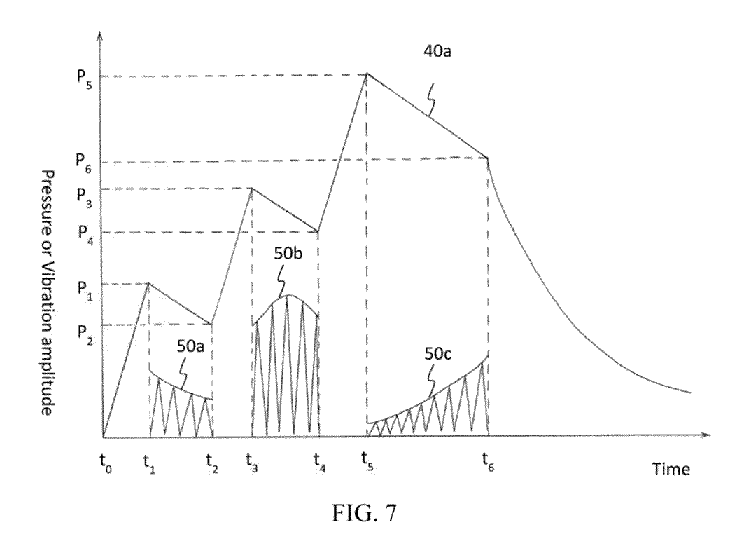

The embodiments in accordance with FIGS. 3, 5 and 6 described as above, detect a pulsation generated from a feedback of the artery at each different pressure value in step S620 and S720, preferably, detecting a pulsation at each different pressure value shall not limited to detect in a single process of inflation or deflation and which is only an alternative embodiment of the present invention. In accordance with the other embodiments, the sphygmogram of pulsation (50) can be obtained by each partial pulsation from the pressure value of the cuff (40) through the staged pressurize way or the staged compression way. For example, FIG. 7 shows another preferred embodiment of a device for detecting signal of pulse diagnosis of the present invention in a detecting output sphygmogram. Please refer to FIG. 7 in combination. In the present embodiment, the cuff (40a) will first pressurize to pressure value P1, and release the pressure to pressure value P2 to obtain a gradually weakened partial sphygmogram of pulsation (50a) during time t1 to t2. Then to pressurize to pressure value to P3, and release the pressure to pressure value P4 to obtain the biggest pulsation of the partial sphygmogram of pulsation (50b) during time t3 to t4. Afterwards, to re-pressurize to pressure value P5, and release the pressure to pressure value P6 to obtain gradually increase partial sphygmogram of pulsation (50c) during time t5 to t6. In other words, the process of detecting pulsation is divided into three stages of pressurization in the present embodiment, but the present invention shall not be limited in this. Those skilled in the art can change the pressurized times and compression times or the regularity of the detecting process as needed.

Accordingly, the present invention is related to a method and a system for detecting signals of pulse diagnosis, and a detecting device of pulse diagnosis. Compare a default sphygmogram model by using a single pulse period and vibration amplitude under different pressure values of the subject, and use the comparison of the sphygmogram as a standard. According to the features of personal pulse condition to determine the applying pressure of pulse diagnosis, fix the pulse collecting position and range of detecting device of pulse diagnosis to collect personal and digitization sphygmogram which is beneficial to do the scientific analysis of TCM pulse condition. Furthermore, there are also other advantages in some embodiments of the present invention exemplarily listed as follows:

1. The method for detecting signals of pulse diagnosis compares personal sphygmogram under different pressures and default sphygmogram models by the graphical analog method to determine the applying pressure of the pulse diagnosis in accordance with the present invention. It is not only to provide the personal pressure of pulse diagnosis, but further meet the position, pace, form, and dynamic of pulse condition in TCM perspective.

2. The detecting device of pulse diagnosis in accordance with the present invention can fix the pulse collecting position, determine the pressure value of pulse diagnosis by comparing the sphygmogram, and digitization the detecting of the sphygmogram to exclude the unquantifiable issues of the human judgment factor causing position difference of pulse taking or the inconsistency of pressing force.

3. The system for detecting signals of pulse diagnosis in accordance with the present invention can comprise the external device which is used to start the detecting device of pulse diagnosis and to receive a sphygmogram detected by the detecting device of pulse diagnosis, and therefore provide the instant and convenient digitization value of pulse diagnosis to the telemedicine doctors.

Although the present invention has been disclosed the embodiments as above, it should be understood those are given by way of illustration only, those skilled in the art will recognize that the present invention can be practiced with modification within the spirit and scope of the claims.

What is claimed is:

1. A method for detecting signals of pulse diagnosis, comprising:
applying a pressure on an artery of an object;
detecting a pulsation generated from a feedback of the artery at each different pressure value;
comparing the similarity of a preset period and a preset vibration amplitude of the pulsation at each different pressure value with a default sphygmogram model to determine a pressure value of pulse diagnosis by a graphical analog method; and
obtaining a corresponding sphygmogram according to the pressure value of pulse diagnosis.

2. The method for detecting signals of pulse diagnosis as claimed in claim 1, wherein the pressure value of pulse diagnosis comprises a pressure value of deep pulse taking, a pressure value of medium pulse taking and a pressure value of floating pulse taking, and the at least two pressure values are determined by changing the other pressure value with a default range.

3. The method for detecting signals of pulse diagnosis as claimed in claim 2, wherein the pressure value of deep pulse taking and the pressure value of floating pulse taking are determined by the pressure value of medium pulse taking adding or subtracting the default range.

4. The method for detecting signals of pulse diagnosis as claimed in claim 3, wherein the default range is 15 mmHg.

5. The method for detecting signals of pulse diagnosis as claimed in claim 3, wherein obtaining a corresponding sphygmogram according to the pressure value of pulse diagnosis is obtained the corresponding sphygmogram from large to small sequentially of the pressure value of pulse diagnosis.

6. The method for detecting signals of pulse diagnosis as claimed in claim 1, wherein obtaining a corresponding sphygmogram is obtaining a corresponding sphygmogram collected from maintaining the pressure value of pulse diagnosis during a default time.

7. The method for detecting signals of pulse diagnosis as claimed in claim 1, wherein the preset period of the pulsation comprises rapid ventricular ejection, left ventricular contraction and left ventricular relaxation.

8. The method for detecting signals of pulse diagnosis as claimed in claim 1, wherein the preset vibration amplitude of the pulsation comprises percussion wave, height of the dicrotic notch and dicrotic wave.

9. The method for detecting signals of pulse diagnosis as claimed in claim 1, further comprising transmitting the sphygmogram to the internet or a data receiver.

10. A detecting device of pulse diagnosis detected a signal of pulse diagnosis by a pulsation generated from an artery of an object, comprising:
a cuff disposed on the object to pressurize while inflation and decompress while deflation;
a detector for detecting the pulsation generated from a feedback of the artery pressurized by the cuff and obtaining a sphygmogram at each different pressure value;
a microprocessor coupled with the detector for comparing the similarity of a preset period and a preset vibration amplitude of the pulsation at each different pressure value with a default sphygmogram model to determine a pressure value of pulse diagnosis by a graphical analog method, and then to obtain a corresponding sphygmogram according to the pressure value of pulse diagnosis.

11. The detecting device of pulse diagnosis as claimed in claim 10, wherein the detecting device of pulse diagnosis is a wrist type detecting device, and the pressure value of pulse diagnosis comprises a pressure value of deep pulse taking, a pressure value of medium pulse taking, and a pressure value of floating pulse taking, and those at least two pressure values are determined by changing the other pressure value with a default range.

12. The detecting device of pulse diagnosis as claimed in claim 11, wherein the pressure value of deep pulse taking and the pressure value of floating pulse taking are determined by the pressure value of medium pulse taking adding or subtracting the default range.

13. The detecting device of pulse diagnosis as claimed in claim 12, wherein the default range is 15 mmHg.

14. The detecting device of pulse diagnosis as claimed in claim 10, wherein the preset period of the pulsation comprises rapid ventricular ejection, left ventricular contraction and left ventricular relaxation.

15. The detecting device of pulse diagnosis as claimed in claim 10, wherein the preset vibration amplitude of the pulsation comprises percussion wave, height of the dicrotic notch and dicrotic wave.

16. The detecting device of pulse diagnosis as claimed in claim 10, further comprising:
an air pump connected to the cuff to pressurize the cuff while inflation;
a controller coupled with the cuff to control the pressure value inside the cuff; and
a pressure sensor coupled with the detector and the microprocessor to convert the pulsation to an electrical signal and provide the preset period and the preset vibration amplitude information of the pulsation.

17. The detecting device of pulse diagnosis as claimed in claim 10, further comprising:
a transmitter coupled with the microprocessor to transmit the corresponding sphygmogram to the internet or a data receiver.

18. The detecting device of pulse diagnosis as claimed in claim 10, further comprising:
a display coupled with the microprocessor to display the sphygmogram and/or the related status information of the pulse diagnosis detecting device.

19. A system for detecting signals of pulse diagnosis, comprising:
a detecting device of pulse diagnosis as claimed in claim 10; and
an external device comprising an actuator and a data receiver, wherein the actuator is used for controlling turning on or off the transmission function of the sphygmogram of the detecting device of pulse diagnosis.

20. A system for detecting signals of pulse diagnosis as claimed in claim 19, wherein the external device is desktop computer, mobile phone, palmtop computer or laptop.
